(19) United States
(12) Patent Application Publication
(10) Pub. No.: US 2024/0384072 A1
(43) Pub. Date: Nov. 21, 2024

(54) COMPOSITION FOR LATEX POLYMERIZATION, LATEX FOR DIP MOLDING, AND DIP MOLDED ARTICLE MANUFACTURED THEREFROM

(71) Applicant: KOREA KUMHO PETROCHEMICAL CO., LTD., Seoul (KR)

(72) Inventors: Jong Jin JEON, Sejong (KR); Duseong AHN, Daejeon (KR); Kun-Ho YANG, Daejeon (KR)

(73) Assignee: KOREA KUMHO PETROCHEMICAL CO., LTD., Seoul (KR)

(21) Appl. No.: 18/691,812

(22) PCT Filed: Sep. 6, 2022
(86) PCT No.: PCT/KR2022/013397

§ 371 Date: Mar. 13, 2024

(30) Foreign Application Priority Data
Sep. 15, 2021 (KR) 10-2021-0122925

(51) Int. Cl. C08L 9/04; B29C 41/14; B29L 31/00; B29L 31/48; C08F 2/26; C08F 2/44; C08F 220/06

(52) U.S. Cl. CPC C08L 9/04; B29C 41/14; C08F 2/26; C08F 2/44; C08F 220/06; B29L 2031/4864; B29L 2031/7538; B29L 2031/7542

(57) ABSTRACT

Disclosed are a composition for latex polymerization including: a conjugated diene-based monomer; an ethylenically unsaturated nitrile monomer, an ethylenically unsaturated acid monomer, and an ionic compound and having an ionic conductivity of 275 μs/cm or more, a latex for dip molding polymerized therefrom, and a dip molded article using the same.

### TECHNICAL FIELD

This specification relates to a composition for latex polymerization, a latex for dip molding polymerized using the same, and a dip molded article manufactured therefrom.

### BACKGROUND ART

Conventionally, dip molded articles such as gloves used for medical, food, inspection, and experimental purposes and condoms were manufactured using natural rubber latex as the main raw material. However, these natural rubber latex molded articles include proteins inside, causing problems such as rashes, itchiness, and colds due to contact allergic reactions in users. As a result, there is an increasing trend in the use of dip molded articles manufactured from synthetic rubber latex as the main raw material, which does not include any protein.

As the use of latex for dip molding increases, there is a need to improve quality, and recent attempts have been made to improve the durability, such as tensile strength and elongation, of a dip molded article manufactured from latex for dip molding. However, despite these attempts to improve mechanical properties, cases of personal accidents or failure to achieve the desired purpose continue to occur due to damage to a dip molded article.

This is because, when the molded article is actually used, its physical properties deteriorate when it comes in contact with slightly acidic body fluids such as human skin or sweat. Conventional mechanical properties, such as tensile strength and elongation, are measured in air at room temperature, and when these properties are excellent, the durability of the dip molded article before use can be guaranteed, but durability under actual use conditions is poor. Therefore, there is a need to develop technology for manufacturing a dip molded article with excellent durability under actual use conditions.

Accordingly, the demand for high-solids latex for dip molding that has excellent quality even in small quantities is also increasing. However, the conventional latex for dip molding has a problem in that when the solid content is increased through concentration, and the like, viscosity increases rapidly above a certain content due to a decrease in particle stability.

In addition, latex for dip molding generally includes particulate polymers with an average particle diameter of around 800 Å. A technology for manufacturing latex including high-quality, large-diameter polymer particles has been proposed by concentrating or enlarging the latex through chemical treatment, and the like, but there is a problem in that the stability of latex is rapidly reduced in the process.

### DISCLOSURE

### Technical Problem

The description in this specification provides a polymerization composition for preparing latex for dip molding that has excellent stability and has a large particle diameter, high solid content, and low viscosity characteristics, and a high-quality dip molded article using the latex for dip molding.

### Technical Solution

According to one aspect, a composition for latex polymerization, which includes: a conjugated diene-based monomer: an ethylenically unsaturated nitrile monomer; an ethylenically unsaturated acid monomer; and an ionic compound and has an ionic conductivity of 275 μs/cm or more, is provided.

In one embodiment, the conjugated diene-based monomer may be one or more selected from the group consisting of 1,3-butadiene, isoprene, 2,3-dimethyl-1,3-butadiene, 1,3-pentadiene, 1,3-hexadiene, 2-phenyl-1,3-butadiene, 3-methyl-1,3-pentadiene, 2-chloro-1,3-butadiene, 3-butyl-1,3-octadiene, and octadiene.

In one embodiment, the ethylenically unsaturated nitrile monomer may be one or more selected from the group consisting of acrylonitrile, methacrylonitrile, fumaronitrile, α-chloronitrile, and α-cyano ethyl acrylonitrile.

In one embodiment, the ethylenically unsaturated acid monomer may be one or more selected from the group consisting of acrylic acid, methacrylic acid, itaconic acid, maleic acid, fumaric acid, maleic anhydride, citraconic anhydride, styrene sulfonic acid, monobutyl fumarate, monobutyl maleate, and mono-2-hydroxypropyl maleate.

In one embodiment, the ionic compound may be one or more selected from the group consisting of sodium chloride, potassium chloride, calcium chloride, magnesium chloride, sodium nitrate, potassium nitrate, calcium nitrate, magnesium nitrate, sodium sulfate, potassium sulfate, calcium sulfate, magnesium sulfate, sodium hydroxide, potassium hydroxide, calcium hydroxide, magnesium hydroxide, sodium bicarbonate, potassium bicarbonate, sodium carbonate, potassium carbonate, sodium bisulfite, potassium bisulfite, sodium pyrophosphate, potassium pyrophosphate, trisodium phosphate, tripotassium phosphate, sodium monohydrogen phosphate, potassium monohydrogen phosphate, ethylenediaminetetraacetic acid or a sodium salt thereof, ethylene glycol tetraacetic acid or a sodium salt thereof, nitrilotriacetic acid or a sodium salt thereof, iminodiacetic acid or a sodium salt thereof, and quinolinic acid or a sodium salt thereof.

In one embodiment, the composition may include 30 to 90 parts by weight of the conjugated diene-based monomer, 1 to 55 parts by weight of the ethylenically unsaturated nitrile monomer, and 0.001 to 20 parts by weight of the ethylenically unsaturated acid monomer.

In one embodiment, the composition may further include water, an emulsifier, a polymerization initiator, and a molecular weight adjusting agent.

According to another aspect, a latex for dip molding, including a copolymer derived from the composition for latex polymerization, is provided, wherein the copolymer has an average particle diameter of 1,000 to 3,000 Å.

In one embodiment, the latex may have a viscosity of 50 to 2,500 cps at 25° C.

In one embodiment, a solid content of the latex may be 50 to 65% by weight.

According to another aspect, a dip molded article manufactured from the latex for dip molding described above is provided.

In one embodiment, the dip molded article may be surgical gloves, medical gloves, gloves for processing agricultural and livestock products, industrial gloves, condoms, cosmetic materials, catheters, or molded articles for health care.

### ADVANTAGEOUS EFFECTS

According to one aspect, when polymerizing latex, the stability of the polymer is significantly improved, and thus the trade-offs of high solid content, large particle diameter, and low viscosity can be simultaneously satisfied.

The effects of one aspect of the present specification is not limited to the above-described effects, and it should be understood to include all effects that can be inferred from the configuration described in the detailed description or claims of the present specification.

### MODES OF THE INVENTION

Hereinafter, one aspect of the present specification will be described based on specific examples. However, the description of this specification may be implemented in several different forms, and thus is not limited to the embodiments described herein.

Throughout the specification, when a part is “connected” to another part, this includes not only the case where it is “directly connected” but also the case where it is “indirectly connected” with another member interposed therebetween. In addition, when a part is said to “include” a component, this means that other components may be further included, not excluded, unless specifically stated otherwise.

When a range of numerical values is recited herein, the values have the precision of the significant figures provided in accordance with the standard rules in chemistry for significant figures, unless a specific range is otherwise stated. For example, 10 includes the range of 5.0 to 14.9, and 10.0 includes the range of 9.50 to 10.49.

### Composition for Latex Polymerization

A composition for latex polymerization according to one aspect may include a conjugated diene-based monomer; an ethylenically unsaturated nitrile monomer; an ethylenically unsaturated acid monomer; and an ionic compound, and have an ionic conductivity of 275 μs/cm or more.

The composition for latex polymerization may be one in which the content of the ionic compound is adjusted to achieve the desired ionic conductivity. For example, the ionic conductivity of the composition may be 275 μs/cm or more, for example, 275 μs/cm, 277.5 μs/cm, 280 μs/cm, 282.5 μs/cm, 285 μs/cm, 287.5 μs/cm, 290 μs/cm, 292.5 μs/cm, 295 μs/cm, 297.5 μs/cm, 300 μs/cm, 302.5 μs/cm, 305 μs/cm, 307.5 μs/cm, 310 μs/cm, 312.5 μs/cm, 315 μs/cm, 317.5 μs/cm, 320 μs/cm, 322.5 μs/cm, 325 μs/cm, 327.5 μs/cm, 330 μs/cm, 332.5 μs/cm, 335 μs/cm, 337.5 μs/cm, 340 μs/cm, 342.5 μs/cm, 345 μs/cm, 347.5 μs/cm, 350 μs/cm, 352.5 μs/cm, 355 μs/cm, 357.5 μs/cm, 360 μs/cm, 362.5 μs/cm, 365 μs/cm, 367.5 μs/cm, 370 μs/cm, 372.5 μs/cm, 375 μs/cm, 377.5 μs/cm, 380 μs/cm, 382.5 μs/cm, 385 μs/cm, 387.5 μs/cm, 390 μs/cm, 392.5 μs/cm, 395 μs/cm, 397.5 μs/cm, 400 μs/cm, a value in a range between two of these values, or one or more of these values. When the ionic conductivity of the composition for latex polymerization is less than the above range, the stability of the polymerized latex may decrease, and in particular, when concentrating the solid content by more than half, viscosity may rapidly increase.

In addition, the ionic conductivity of the composition may be 10 ms/cm or less, 5 ms/cm or less, or 1 ms/cm or less, but is not limited thereto. When the ionic conductivity of the composition is excessively high, components unnecessary for polymerization may increase, and thus the stability of the latex may decrease.

The stability of latex may be improved by controlling the ionic conductivity during polymerization of latex including copolymers derived from conjugated diene-based monomers, ethylenically unsaturated nitrile monomers, and ethylenically unsaturated acid monomers. Using this, low viscosity may be maintained even at a high solid content of 50% by weight or more. In addition, stability is maintained, and a copolymer with a large particle diameter of 1,000 Å or more may be formed.

The conjugated diene-based monomer may be one or more selected from the group consisting of 1,3-butadiene, isoprene, 2,3-dimethyl-1,3-butadiene, 1,3-pentadiene, 1,3-hexadiene, 2-phenyl-1,3-butadiene, 3-methyl-1,3-pentadiene, 2-chloro-1,3-butadiene, 3-butyl-1,3-octadiene, and octadiene. In the copolymer of latex for dip molding, the structure derived from the conjugated diene-based monomer may provide flexibility to a dip molded article.

The ethylenically unsaturated nitrile monomer may be one or more selected from the group consisting of acrylonitrile, methacrylonitrile, fumaronitrile, α-chloronitrile, and α-cyano ethyl acrylonitrile. In the copolymer of latex for dip molding, the structure derived from the ethylenically unsaturated nitrile monomer may improve the strength and chemical resistance of the dip molded article.

The ethylenically unsaturated acid monomer may be one or more selected from the group consisting of acrylic acid, methacrylic acid, itaconic acid, maleic acid, fumaric acid, maleic anhydride, citraconic anhydride, styrene sulfonic acid, monobutyl fumarate, monobutyl maleate, and mono-2-hydroxypropyl maleate. In the copolymer of latex for dip molding, the structure derived from the ethylenically unsaturated acid monomer forms a cross-linked structure and thus may improve the mechanical properties of the dip molded article.

The ionic compound may be one or more selected from the group consisting of sodium chloride (NaCl), potassium chloride (KCl), calcium chloride (CaCl₂), magnesium chloride (MgCl₂), sodium nitrate (NaNO₃), potassium nitrate (KNO₃), calcium nitrate (Ca(NO₃)₂), magnesium nitrate (Mg(NO₃)₂), sodium sulfate (Na₂SO₄), potassium sulfate (K₂SO₄), calcium sulfate (CaSO₄), magnesium sulfate (MgSO₄), sodium hydroxide (NaOH), potassium hydroxide (KOH), calcium hydroxide (Ca(OH)₂), magnesium hydroxide (Mg(OH)₂), sodium bicarbonate (NaHCO₃), potassium bicarbonate (KHCO₃), sodium carbonate (Na₂CO₃), potassium carbonate (K₂CO₃), sodium bisulfite (NaHSO₄), potassium bisulfite (KHSO₄), sodium pyrophosphate (Na₄P₂O₇), potassium pyrophosphate (K₄P₂O₇), trisodium phosphate (Na₃PO₄), tripotassium phosphate (K₃PO₄), sodium monohydrogen phosphate (Na₂HPO₄), potassium monohydrogen phosphate (K₂HPO₄), ethylenediaminetetraacetic acid (EDTA) or a sodium salt thereof, ethylene glycol tetraacetic acid (EGTA) or a sodium salt thereof, nitrilotriacetic acid (NTA) or a sodium salt thereof, iminodiacetic acid (IDA) or a sodium salt thereof, and quinolinic acid (QNA) or a sodium salt thereof. The ionic compound may improve the stability of the copolymer while the reaction is in progress during the copolymerization of latex for dip molding and may suppress aggregation of the latex even after polymerization.

The composition may include 30 to 90 parts by weight of the conjugated diene-based monomer, 1 to 55 parts by weight of the ethylenically unsaturated nitrile monomer, and 0.001 to 20 parts by weight of the ethylenically unsaturated acid monomer.

For example, the conjugated diene-based monomer content of the composition may be 30 parts by weight, 32.5 parts by weight, 35 parts by weight, 37.5 parts by weight, 40 parts by weight, 42.5 parts by weight, 45 parts by weight, 47.5 parts by weight, 50 parts by weight, 52.5 parts by weight, 55 parts by weight, 57.5 parts by weight, 60 parts by weight, 62.5 parts by weight, 65 parts by weight, 67.5 parts by weight, 70 parts by weight, 72.5 parts by weight, 75 parts by weight, 77.5 parts by weight, 80 parts by weight, 82.5 parts by weight, 85 parts by weight, 87.5 parts by weight, 90 parts by weight, or a value in a range between two of these values. When the content of the conjugated diene-based monomer is less than 30 parts by weight, the dip molded article may be excessively hardened, resulting in a poor fit, and when the content is more than 90 parts by weight, the durability or chemical resistance of the dip molded article may be reduced.

The ethylenically unsaturated nitrile monomer content of the composition may be 1 part by weight, 2.5 parts by weight, 5 parts by weight, 7.5 parts by weight, 10 parts by weight, 12.5 parts by weight, 15 parts by weight, 17.5 parts by weight, 20 parts by weight, 22.5 parts by weight, 25 parts by weight, 27.5 parts by weight, 30 parts by weight, 32.5 parts by weight, 35 parts by weight, 37.5 parts by weight, 40 parts by weight, 42.5 parts by weight, 45 parts by weight, 47.5 parts by weight, 50 parts by weight, 52.5 parts by weight, 55 parts by weight, or a value in a range between two of these values. When the content of the ethylenically unsaturated nitrile monomer is less than 1 part by weight, the chemical resistance or mechanical strength of the dip molded article may be reduced, and when the content exceeds 55 parts by weight, the elongation of the dip molded article may be reduced, and thus usability may be reduced.

The ethylenically unsaturated acid monomer content of the composition may be 0.001 parts by weight, 0.5 parts by weight, 1 part by weight, 1.5 parts by weight, 2 parts by weight, 2.5 parts by weight, 3 parts by weight, 3.5 parts by weight, 4 parts by weight, 4.5 parts by weight, 5 parts by weight, 5.5 parts by weight, 6 parts by weight, 6.5 parts by weight, 7 parts by weight, 7.5 parts by weight, 8 parts by weight, 8.5 parts by weight, 9 parts by weight, 9.5 parts by weight, 10 parts by weight, 10.5 parts by weight, 11 parts by weight, 11.5 parts by weight, 12 parts by weight, 12.5 parts by weight, 13 parts by weight, 13.5 parts by weight, 14 parts by weight, 14.5 parts by weight, 15 parts by weight, 15.5 parts by weight, 16 parts by weight, 16.5 parts by weight, 17 parts by weight, 17.5 parts by weight, 18 parts by weight, 18.5 parts by weight, 19 parts by weight, 19.5 parts by weight, 20 parts by weight, or a value in a range between two of these values. When the content of the ethylenically unsaturated acid monomer is less than 0.001 parts by weight, the tensile strength of the dip molded article may decrease, and when the content exceeds 20 parts by weight, the dip molded article may harden excessively, resulting in a poor fit.

In this specification, “total monomers” means the sum of the conjugated diene-based monomer, the ethylenically unsaturated nitrile monomer, and the ethylenically unsaturated acid monomer, but the composition for latex polymerization may further include polymerizable monomers other than the above-described conjugated diene-based monomer, ethylenically unsaturated nitrile monomer, and ethylenically unsaturated acid monomer, as long as the durability test result does not exceed the above range, and at this time, the “total monomers” further includes the polymerizable monomers.

In a non-limiting example, the monomers included in the composition may be composed of the above-described conjugated diene-based monomer, ethylenically unsaturated nitrile monomer, and ethylenically unsaturated acid monomer. When copolymerizing the above three types of monomers, the stability of latex may be improved and the average particle diameter may be increased by controlling ionic conductivity through the above-described ionic compounds, but it may be difficult to realize these effects when additional monomers are added.

The ionic compound content may vary depending on a monomer composition ratio and the type of ionic compound. The ionic compound content may be 0.1 to 5 parts by weight, for example, 0.1 parts by weight, 0.5 parts by weight, 1 part by weight, 1.5 parts by weight, 2 parts by weight, 2.5 parts by weight, 3 parts by weight, 3.5 parts by weight, 4 parts by weight, 4.5 parts by weight, 5 parts by weight, or a value in a range between two of these values, based on 100 parts by weight of the total monomers under conditions that satisfy the above-described ionic conductivity. When the content is outside the above range, it may be difficult to satisfy the ionic conductivity condition, or even when the ionic conductivity is satisfied, a stability improvement effect may not be realized.

The composition for latex polymerization may further include water, an emulsifier, a polymerization initiator, and a molecular weight adjusting agent.

For example, the water content may be 75 to 150 parts by weight, for example, 75 parts by weight, 77.5 parts by weight, 80 parts by weight, 82.5 parts by weight, 85 parts by weight, 87.5 parts by weight, 90 parts by weight, 92.5 parts by weight, 95 parts by weight, 97.5 parts by weight, 100 parts by weight, 102.5 parts by weight, 105 parts by weight, 107.5 parts by weight, 110 parts by weight, 112.5 parts by weight, 115 parts by weight, 117.5 parts by weight, 120 parts by weight, 122.5 parts by weight, 125 parts by weight, 127.5 parts by weight, 130 parts by weight, 132.5 parts by weight, 135 parts by weight, 137.5 parts by weight, 140 parts by weight, 142.5 parts by weight, 145 parts by weight, 147.5 parts by weight, 150 parts by weight, or a value in a range between two of these values, based on 100 parts by weight of the total monomers. When the content of water is less than 75 parts by weight, viscosity excessively increases during polymerization, making it difficult to manufacture molded articles, and when the content is more than 150 parts by weight, the solid content may be excessively low. The water may have an ionic conductivity of 5 μs/cm or less, 2.5 μs/cm or less, or 1 μs/cm or less. For example, the water may be ion-exchanged water, ultrapure water, or purified water. When water with high ionic conductivity is used, it may contain impurities that adversely affect polymerization stability or latex stability.

The emulsifier may be an anionic surfactant, a nonionic surfactant, a cationic surfactant, or an amphoteric surfactant. For example, as the anionic surfactant, one or more selected from the group consisting of alkylbenzene sulfonates, aliphatic sulfonates, sulfuric acid ester salts of higher alcohols, α-olefin sulfonate salts, and alkyl ether sulfuric acid ester salts may be used, but are not limited thereto. The emulsifier may be used in an amount of 0.8 to 8 parts by weight based on 100 parts by weight of the total monomers. Depending on the content of the emulsifier, the ionic compound content may vary to achieve the above-described ionic conductivity.

The polymerization initiator may be a radical initiator. The radical initiator may be, for example, one or more of an inorganic peroxide selected from the group consisting of sodium persulfate, potassium persulfate, ammonium persulfate, potassium perphosphate, and hydrogen peroxide: an organic peroxide selected from the group consisting of t-butyl peroxide, cumene hydroperoxide, p-menthane hydroperoxide, di-t-butyl peroxide, t-butylcumyl peroxide, acetyl peroxide, isobutyl peroxide, octanoyl peroxide, dibenzoyl peroxide, 3,5,5-trimethylhexanol peroxide, and t-butylperoxyisobutyrate: and an azo-based initiator selected from the group consisting of azobisisobutyronitrile, azobis-2,4-dimethylvaleronitrile, azobiscyclohexanecarbonitrile, and methyl azobisisobutyric acid (butyric acid), but is not limited thereto. The polymerization initiator may be used in an amount of 0.01 to 1.5 parts by weight based on 100 parts by weight of the total monomers.

The molecular weight adjusting agent may be mercaptans such as an α-methylstyrene dimer, t-dodecyl mercaptan, n-dodecyl mercaptan, and octyl mercaptan: halogenated hydrocarbons such as carbon tetrachloride, methylene chloride, and methylene bromide; and sulfur-containing compounds such as tetraethylthiuram disulfide, dipentamethylenethiuram disulfide, and diisopropyl xanthogen disulfide, but is not limited thereto. The molecular weight adjusting agent content may be 0.1 to 1 part by weight based on 100 parts by weight of the total monomers of the copolymer latex. For example, the molecular weight adjusting agent may be 0.1 parts by weight, 0.15 parts by weight, 0.2 parts by weight, 0.25 parts by weight, 0.3 parts by weight, 0.35 parts by weight, 0.4 parts by weight, 0.45 parts by weight, 0.5 parts by weight, 0.55 parts by weight, 0.6 parts by weight, 0.65 parts by weight, 0.7 parts by weight, 0.75 parts by weight, 0.8 parts by weight, 0.85 parts by weight, 0.9 parts by weight, 0.95 parts by weight, 1 part by weight, or a value in a range between two of these values. When the content of the molecular weight adjusting agent is less than 0.1 part by weight, a gel may be formed, and thus latex stability may be reduced, and when the content is more than 1 part by weight, tensile strength may be poor, stress retention may be reduced, and durability in actual use may be reduced.

### Latex for Dip Molding

The latex for dip molding according to another aspect is a latex for dip molding including a copolymer derived from the composition for latex polymerization described above, and an average particle diameter of the copolymer may be 1,000 to 3,000 Å, for example, 1,000 Å, 1,050 Å, 1,100 Å, 1,150 Å, 1,200 Å, 1,250 Å, 1,300 Å, 1,350 Å, 1,400 Å, 1,450 Å, 1,500 Å, 1,550 Å, 1,600 Å, 1,650 Å, 1,700 Å, 1,750 Å, 1,800 Å, 1,850 Å, 1,900 Å, 1,950 Å, 2,000 Å, 2,050 Å, 2,100 Å, 2,150 Å, 2,200 Å, 2,250 Å, 2,300 Å, 2,350 Å, 2,400 Å, 2,450 Å, 2,500 Å, 2,550 Å, 2,600 Å, 2,650 Å, 2,700 Å, 2,750 Å, 2,800 Å, 2,850 Å, 2,900 Å, 2,950 Å, 3,000 Å, or a value in a range between two of these values. The latex for dip molding is polymerized while controlling ionic conductivity, thereby minimizing the decrease in stability and enlarging the copolymer particle diameter. In addition, the latex may have better stability due to its low oligomer content.

The latex for dip molding may have a zeta potential (absolute value) of 60 mV or more, 62.5 mV or more, 65 mV or more, 67.5 mV or more, or 70 mV or more. Latex that satisfies these conditions has excellent stability and thus may suppress an increase in viscosity even when concentrated with a high solid content of 50% by weight or more.

The viscosity of the latex for dip molding at 25° C. may be 50 to 2,500 cps, for example, 50 cps, 75 cps, 100 cps, 125 cps, 150 cps, 175 cps, 200 cps, 225 cps, 250 cps, 275 cps, 300 cps, 325 cps, 350 cps, 375 cps, 400 cps, 425 cps, 450 cps, 475 cps, 500 cps, 525 cps, 550 cps, 575 cps, 600 cps, 625 cps, 650 cps, 675 cps, 700 cps, 725 cps, 750 cps, 775 cps, 800 cps, 825 cps, 850 cps, 875 cps, 900 cps, 925 cps, 950 cps, 975 cps, 1,000 cps, 1,100 cps, 1,200 cps, 1,300 cps, 1,400 cps, 1,500 cps, 1,600 cps, 1,700 cps, 1,800 cps, 1,900 cps, 2,000 cps, 2,100 cps, 2,200 cps, 2,300 cps, 2,400 cps, 2,500 cps, or a value in a range between two of these values. Latex with a viscosity outside the above range may be practically impossible to manufacture or may be difficult to dip mold.

The solid content of the latex for dip molding may be 50 to 65% by weight, for example, 50% by weight, 52.5% by weight, 55% by weight, 57.5% by weight, 60% by weight, 62.5% by weight, 65% by weight, or a value in a range between two of these values. When the solid content is outside the above range, the effect of improving stability described above may be unnecessary or aggregation of latex may occur.

The characteristics of the latex for dip molding may be measured at pH 8.0 to 10.0, for example, pH 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0. When adjusting the pH of latex through additives, the solid content and average particle diameter may change, but the latex for dip molding may simultaneously meet the above-described average particle diameter, solid content, and viscosity requirements in the above pH range.

The latex for dip molding may further include one or more additives selected from the group consisting of chelating agents, dispersants, pH adjusters, deoxidizers, particle diameter adjusters, anti-aging agents, and oxygen trapping agents. As these additives, compositions known in the art can be used, and as long as they satisfy the above-described ionic conductivity range, descriptions of the type, function, and addition amount will be omitted. These additives may be added before or after polymerization of the copolymer.

The latex for dip molding satisfies low viscosity, high solid content, and large particle diameter at the same time, so the latex itself may have excellent stability. Therefore, it is possible to prevent the quality of a dip molded article from deteriorating even when external shock is applied or when stored for a long period of time.

### Method of Preparing Latex for Dip Molding

The method of preparing a latex for dip molding includes: (a) adding an emulsifier and water to a monomer mixture including a conjugated diene-based monomer, an ethylenically unsaturated nitrile monomer, and an ethylenically unsaturated acid monomer: (b) adjusting ionic conductivity by adding an ionic compound: and (c) preparing a latex for dip molding by adding a polymerization initiator, wherein in step (b), the ionic compound may be added so that ionic conductivity is 275 μs/cm or more.

Step (a) is a step of preparing a monomer mixture including a conjugated diene-based monomer, an ethylenically unsaturated nitrile monomer, and an ethylenically unsaturated acid monomer, which are monomers constituting a carboxylic acid-modified nitrile-based copolymer, and adding the mixture to emulsified water, and may performed under a nitrogen atmosphere.

In step (a) or (b), a molecular weight adjusting agent or an emulsifier may be further added. These are the same as described above.

Step (b) is a step of preparing the composition for latex polymerization described above by adding an ionic compound, and may realize the above-mentioned stability improvement effect by controlling ionic conductivity after adding all the components necessary for polymerization.

The polymerization in step (c) may be performed at 10 to 90° C., for example, 10° C., 15 ° C., 20° C., 25° C., 30° C., 35° C., 40° C., 45° C., 50° C., 55° C., 60° C., 65° C., 70° C., 75° C., 80° C., 85° C., 90° C., or a temperature between two of these temperatures.

When a conversion rate of the polymerization reaction is 90% or more, the polymerization may be stopped by adding a polymerization terminator. For example, the polymerization terminator may be added when a conversion rate of the polymerization reaction is 90%, 91%, 92%, 93%, 94%, 95%, 96%, 97%, 98%, 99%, 99.9%, or a value between two of these values.

The polymerization terminator may be one selected from the group consisting of hydroxyl amine, hydroxy amine sulfate, diethyl hydroxy amine, hydroxy amine sulfonic acid and alkali metal ions thereof, sodium dimethyldithiocarbamate, hydroquinone derivatives, aromatic hydroxy dithio carboxylic acids such as hydroxy diethyl benzene dithio carboxylic acid, hydroxy dibutyl benzene dithio carboxylic acid, and the like, and a combination of two or more thereof. The content of the polymerization terminator may be 0.02 to 1.5 parts by weight based on 100 parts by weight of the monomer mixture.

Other raw materials and contents used in the above preparation method are as described above.

### Dip Molded Article

A dip molded article according to another aspect may be manufactured using the latex for dip molding described above.

The dip molded article may be manufactured by adding 0.1 to 1 part by weight of zinc oxide, 1 to 2 parts by weight of sulfur, and 0.3 to 1.5 parts by weight of a vulcanization accelerator to the latex for dip molding described above, based on 100 parts by weight of the copolymer, and then dip molding the mixture.

The zinc oxide may form a cross-linked structure by forming an ionic bond with a structure derived from the ethylenically unsaturated acid. In addition, when the ionic conductivity range described above is satisfied using an ionic compound, durability may be enhanced by improving the ionic bond strength. The content of the zinc oxide may be, for example, 0.1 parts by weight, 0.2 parts by weight, 0.3 parts by weight, 0.4 parts by weight, 0.5 parts by weight, 0.6 parts by weight, 0.7 parts by weight, 0.8 parts by weight, 0.9 parts by weight, or 1 part by weight. When the content of the zinc oxide is excessively small, durability in actual use may be reduced, and when the content is excessively large, tensile strength may be reduced.

The sulfur may react with a structure derived from the conjugated diene-based monomer to form a cross-linked structure. When the ionic conductivity range described above is satisfied using the ionic compound, molded article shrinkage due to syneresis during vulcanization may be suppressed. The content of the sulfur may be, for example, 1 part by weight, 1.1 parts by weight, 1.2 parts by weight, 1.3 parts by weight, 1.4 parts by weight, 1.5 parts by weight, 1.6 parts by weight, 1.7 parts by weight, 1.8 parts by weight, 1.9 parts by weight, or 2.0 parts by weight. When the content of the sulfur is less than 1 part by weight, mechanical properties such as tensile strength and durability in actual use may be reduced, and when the content is more than 2 parts by weight, an allergic reaction may occur in users.

The dip molded article may be dip molded after adjusting the solid content by adding an aqueous potassium hydroxide solution to the latex for dip molding, but is not limited thereto.

The tensile strength of the dip molded article may be 3 MPa or more, 5 MPa or more, 7 MPa or more, 9 MPa or more, 11 MPa or more, 13 MPa or more, 15 MPa or more, 20 MPa or more, 25 MPa or more, 30 MPa or more, or 35 MPa or more, but is not limited thereto. The higher the tensile strength, the better the durability during storage, but other mechanical properties such as elongation may decrease.

The elongation of the dip molded article may be 600% or more, 650% or more, 700% or more, 750% or more, 800% or more, 850% or more, or 900% or more, but is not limited thereto. The higher the elongation, the better the fit, but there may be a trade-off with other mechanical properties.

The dip molded article may have a durability test result of 60 minutes or more, a tensile strength of 10 MPa or more, and an elongation of 600% or more. By controlling ionic conductivity during polymerization, a high-quality dip molded article may be manufactured by maintaining minimum tensile strength and elongation and at the same time having excellent durability in actual use as confirmed by durability tests.

### DURABILITY TEST METHOD

The following process was repeated to measure the time at which the molded product broke: a dip molded article with a width of 30 mm, a length of 135 mm, and a thickness of 0.06 to 0.08 mm is stretched 20% longitudinally, immersed in a pH 4.0 to 4.3 solution at 35° C., and stretched for 10 seconds to achieve a longitudinal elongation of 50%, and after being fixed for 2 seconds, relaxed for 10 seconds so that the longitudinal elongation is 20%.

The durability test method is to check whether the specimen is broken by repeating stretching and relaxation in a solution of pH 4.0 to 4.3 at 35° C., which is similar to skin and body fluids that are likely to come into contact with the dip molded article in actual use, for example, when the molded article is a glove, the durability of the dip molded article under actual use conditions may be measured by simulating a situation in which the molded article repeats stretching and relaxation according to the movement of the finger.

The dip molded article may be surgical gloves, medical gloves, gloves for processing agricultural and livestock products, industrial gloves, condoms, cosmetic materials, catheters, or molded articles for health care.

For example, the dip molded article may be surgical gloves or other medical gloves, industrial gloves such as gloves for handling chemicals, or cosmetic materials such as puffs, but is not limited thereto.

Hereinafter, examples of the present specification will be described in more detail. However, the following experimental results describe only representative experimental results among the examples, and the scope and content of the present invention may not be interpreted as being reduced or limited by the examples. Each effect of various implementations of the present invention not explicitly presented below will be specifically described in the corresponding section. Unless otherwise stated, each test may be performed at 20° C. and 1 atm.

### EXAMPLES AND COMPARATIVE EXAMPLES

A 1 L high-pressure reactor equipped with a stirrer, thermometer, cooler, and nitrogen gas inlet and equipped to continuously introduce each component such as a monomer, an emulsifier, and a polymerization initiator was prepared. Ion-exchanged water with a conductivity of 1 μs/cm or less was prepared. After the atmosphere in the reactor was replaced with nitrogen, a monomer mixture of 74 parts by weight of isoprene (IPM), 24 parts by weight of acrylonitrile (AN), and 2 parts by weight of methacrylic acid (MAA) was input. Then, based on 100 parts by weight of the monomer mixture, an ionic compound (IC), 0.5 parts by weight of t-dodecyl mercaptan as a molecular weight adjusting agent, 2 parts by weight of sodium alkylbenzenesulfonate as an emulsifier, and 120 parts by weight of ion-exchanged water were input to the reactor. The ionic conductivity of the reactants was measured, and an ionic compound was input until the desired ionic conductivity was reached to prepare a composition for latex polymerization. After raising the temperature of the reactor to about 25° C., 0.3 parts by weight of potassium persulfate was input. The types of ionic compounds used in each Example and Comparative Example and the ionic conductivity of the composition for latex polymerization are shown in Table 1 below.

When a conversion rate reached about 95%, 0.9 parts by weight of sodium hydroxide was input to stop the polymerization reaction. Thereafter, unreacted monomers and the like were removed through a deodorizing process, and ammonia water, antioxidants, antifoaming agents, and the like were added to obtain a carboxylic acid-modified nitrile-based copolymer latex of pH 8.5 (however, pH 9.6 in Example 6 and Comparative Example 6). The zeta potential, average particle diameter, solid content, and viscosity of the latex were measured and shown in Table 1 below.

In Comparative Example 7, no ionic compound was added to achieve the desired ionic conductivity, and 0.05 parts by weight of a molecular weight adjusting agent and 0.1 parts by weight of an emulsifier were input, but polymerization was not possible.

TABLE 1

| | | | | Average | Solid | |
|---|---|---|---|---|---|---|
| | | Ionic | Zeta | particle | content | 25° C. |
| | | conductivity | potential | diameter | (% by | viscosity |
| Classification | IC | (μs/cm) | (mV) | (Å) | weight) | (cps) |
| Example 1 | K₂CO₃ | 297.3 | −74.7 | 1,160 | 53.2 | 230 |
| Example 2 | Na₂CO₃ | 332.5 | −81.2 | 1,200 | 54.5 | 420 |
| Example 3 | K₂SO₄ | 298.5 | −75.2 | 1,010 | 50.3 | 110 |
| Example 4 | Na₂SO₄ | 292.5 | −72 | 1,330 | 50.1 | 150 |
| Example 5 | Na₂HPO₄ | 293 | −71.3 | 1,520 | 54.9 | 250 |
| Example 6 | EGTA | 292.8 | −73.1 | 1,950 | 57.7 | 150 |
| Example 7 | EDTA | 311.2 | −78.4 | 1,130 | 52.5 | 170 |
| Example 8 | NTA | 296.5 | −75.3 | 1,160 | 53.3 | 220 |
| Example 9 | DTPA | 325.7 | −79.3 | 1,080 | 50.4 | 95 |
| Example 10 | IDA | 293.2 | −75.9 | 1,210 | 54.7 | 480 |
| Comparative | K₂CO₃ | 274 | −59.8 | 880 | 33.1 | 315 |
| Example 1 | | | | | | |
| Comparative | K₂CO₃ | 269 | −59.3 | 880 | 54.9 | 5,300 |
| Example 2 | | | | | | |
| Comparative | EDTA | 262 | −59.7 | 1,180 | 55.5 | 8,800 |
| Example 3 | | | | | | |
| Comparative | — | 243 | −54 | 920 | 49.2 | 105 |
| Example 4 | | | | | | |
| Comparative | — | 232.6 | −53.9 | 970 | 55.6 | >10,000 |
| Example 5 | | | | | | |
| Comparative | — | 239.8 | −54.1 | 990 | 57.3 | >10,000 |
| Example 6 | | | | | | |
| Comparative | — | 184.2 | — | — | — | — |
| Example 7 | | | | | | |

Ionic conductivity (μs/cm): Ionic conductivity was determined by the Nyquist method after measuring resistance using electrochemical impedance spectroscopy (EIS) using a two-electrode method. Resistance was measured under the conditions of a frequency of 60 Hz to 1 kHz, a current of 10.0 mV, and a voltage range of ±10 V. Ionic conductivity was measured after correction with a standard solution in the expected range. A graphite electrode was used as an electrode. For ionic conductivity, the corrected value based on 25° C. was used.
Zeta potential (mV): Zeta potential at 25° C. was measured using a Malvern Zetasizer instrument.
Average particle diameter (Å): It was measured by dynamic laser light scattering using Nanotrac 150.
25° C. viscosity (cps): It was measured using a Brookfield viscometer using spindle 62 at a spindle speed of 100 rpm.

Referring to Table 1, in the case of the examples where the ionic conductivity of the composition for latex polymerization was relatively high, the zeta potential, which indicates the electrical stability of the latex particles, was measured to be relatively large. As a result, it is judged that it is possible to have low viscosity while achieving a large particle diameter of 1,000 Å or more and a high solid content of 50% by weight or more. On the other hand, the zeta potential of the latex of the comparative examples polymerized under conditions of low ionic conductivity was measured to be relatively small. Therefore, due to lack of stability, it was difficult to achieve a large particle diameter, high solid content, and low viscosity at the same time. Particularly, the latex of the comparative examples with a small particle diameter had a problem in that viscosity rapidly increased when the solid content was increased through concentration. In addition, when polymerization is performed while controlling ionic conductivity, the production of low molecular weight oligomers is expected to decrease, thereby increasing the stability of latex.

### Experimental Examples

To 100 parts by weight of the latex of the examples and comparative examples, 1.8 parts by weight of sulfur(S), 0.7 parts by weight of zinc oxide (ZnO) and 1.2 parts by weight of zinc dibutyldithiocarbamate (ZDBC) as a vulcanization accelerator were added. Thereafter, an aqueous 4% potassium hydroxide solution and double distilled water were added to prepare a composition for dip molding with a solid content concentration of 20% and a pH of 10.0. Rectangular specimens with a width of 30 mm, a length of 135 mm, and a thickness of 0.06 to 0.08 mm were prepared with the dip molding composition, and physical properties were measured and shown in Table 2 below.

TABLE 2

| | Tensile strength | Elongation | S/R | Durability |
|---|---|---|---|---|
| Classification | (MPa) | (%) | (%) | (min) |
| Example 4 | 26.8 | 729 | 34.1 | >180 |
| Example 5 | 34.2 | 722 | 31.4 | >180 |
| Example 6 | 35.7 | 660 | 38.8 | >180 |
| Comparative | 28.9 | 575 | 27.0 | 153 |
| Example 1 | | | | |
| Comparative | 25.7 | 608 | 28.4 | 164 |
| Example 4 | | | | |

Tensile strength and elongation: Using a universal testing machine (UTM), a dumbbell-shaped specimen was stretched at a rate of 500 mm/min, and the tensile load and elongation applied when fracture of the specimen occurred were measured.
Stress retention (S/R): Using a universal testing machine, the specimen was stretched to 100% elongation at a rate of 500 mm/min, and then the initial tensile load (σ0) was measured, the tensile load (σ6) after 6 minutes was measured, and the stress retention was calculated according to the following equation.

$$S / R ( \% ) = \frac{{σ}_{6}}{{σ}_{0}} \times 100$$

Durability: A solution of pH 4 was prepared using citric acid and maintained at 35° C. A rectangular specimen was input into the solution while being stretched 20% in the longitudinal direction. The specimen was stretched to an elongation of 50% for 10 seconds, fixed for 2 seconds, and then relaxed to an elongation of 20% for 10 seconds, and the time at which the specimen was broken was measured.

Referring to Table 2, the specimens manufactured using the latex of the Examples were superior to the Comparative Examples in both mechanical strength and durability. In particular, when comparing Example 4 and Comparative Example 4, which had similar solid contents, the specimen of Example 4, which had relatively large average particles, had excellent mechanical strength and durability. This is believed to be because shrinkage of the film occurred in Comparative Example 4, which had a small average particle diameter, when manufacturing specimens through dip molding.

The description of the present specification described above is for illustrative purposes, and it should be understood that those of ordinary skill in the art to which one aspect of the present specification belongs can easily modify it into other specific forms without changing the technical idea or essential features described in this specification. Therefore, it should be understood that the embodiments described above are illustrative in all respects and not restrictive. For example, each component described as a single type may be implemented in a distributed form, and likewise components described as distributed may be implemented in a combined form.

The scope of the present specification is indicated by the following claims, and all changes or modifications derived from the meaning and scope of the claims and their equivalents should be construed as being included in the scope of the present specification.

1. A composition for latex polymerization comprising:
a conjugated diene-based monomer;
an ethylenically unsaturated nitrile monomer;
an ethylenically unsaturated acid monomer; and
an ionic compound,
wherein an ionic conductivity is 275 μs/cm or more.

2. The composition of claim 1, wherein the conjugated diene-based monomer is one or more selected from the group consisting of 1,3-butadiene, isoprene, 2,3-dimethyl-1,3-butadiene, 1,3-pentadiene, 1,3-hexadiene, 2-phenyl-1,3-butadiene, 3-methyl-1,3-pentadiene, 2-chloro-1,3-butadiene, 3-butyl-1,3-octadiene, and octadiene.

3. The composition of claim 1, wherein the ethylenically unsaturated nitrile monomer is one or more selected from the group consisting of acrylonitrile, methacrylonitrile, fumaronitrile, α-chloronitrile, and α-cyano ethyl acrylonitrile.

4. The composition of claim 1, wherein the ethylenically unsaturated acid monomer is one or more selected from the group consisting of acrylic acid, methacrylic acid, itaconic acid, maleic acid, fumaric acid, maleic anhydride, citraconic anhydride, styrene sulfonic acid, monobutyl fumarate, monobutyl maleate, and mono-2-hydroxypropyl maleate.

5. The composition of claim 1, wherein the ionic compound is one or more selected from the group consisting of sodium chloride, potassium chloride, calcium chloride, magnesium chloride, sodium nitrate, potassium nitrate, calcium nitrate, magnesium nitrate, sodium sulfate, potassium sulfate, calcium sulfate, magnesium sulfate, sodium hydroxide, potassium hydroxide, calcium hydroxide, magnesium hydroxide, sodium bicarbonate, potassium bicarbonate, sodium carbonate, potassium carbonate, sodium bisulfite, potassium bisulfite, sodium pyrophosphate, potassium pyrophosphate, trisodium phosphate, tripotassium phosphate, sodium monohydrogen phosphate, potassium monohydrogen phosphate, ethylenediaminetetraacetic acid or a sodium salt thereof, ethylene glycol tetraacetic acid or a sodium salt thereof, nitrilotriacetic acid or a sodium salt thereof, iminodiacetic acid or a sodium salt thereof, and quinolinic acid or a sodium salt thereof.

6. The composition of claim 1, wherein the composition includes 30 to 90 parts by weight of the conjugated diene-based monomer, 1 to 55 parts by weight of the ethylenically unsaturated nitrile monomer, and 0.001 to 20 parts by weight of the ethylenically unsaturated acid monomer.

7. The composition of claim 1, further comprising water, an emulsifier, a polymerization initiator, and a molecular weight adjusting agent.

8. A latex for dip molding comprising a copolymer derived from the composition for latex polymerization of claim 1, wherein the copolymer has an average particle diameter of 1,000 to 3,000 Å.

9. The latex of claim 8, wherein the latex has a viscosity of 50 to 2,500 cps at 25° C.

10. The latex of claim 8, wherein a solid content of the latex is 50 to 65% by weight.

11. A dip molded article manufactured from the latex for dip molding of claims 8.

12. The dip molded article of claim 11, wherein the dip molded article is surgical gloves, medical gloves, gloves for processing agricultural and livestock products, industrial gloves, condoms, cosmetic materials, catheters, or molded articles for health care.

13. A dip molded article manufactured from the latex for dip molding of claim 9.

14. A dip molded article manufactured from the latex for dip molding of claim 10.

15. The dip molded article of claim 13, wherein the dip molded article is surgical gloves, medical gloves, gloves for processing agricultural and livestock products, industrial gloves, condoms, cosmetic materials, catheters, or molded articles for health care.

16. The dip molded article of claim 14, wherein the dip molded article is surgical gloves, medical gloves, gloves for processing agricultural and livestock products, industrial gloves, condoms, cosmetic materials, catheters, or molded articles for health care.
